(19) United States
(12) Patent Application Publication
(10) Pub. No.: US 2005/0113455 A1
(43) Pub. Date: May 26, 2005

(54) Method and composition for amelioration of pain

(76) Inventor: Raymond J. Bergeron JR., Gainsville, FL (US)

Correspondence Address: MILES & STOCKBRIDGE PC, 1751 PINNACLE DRIVE, SUITE 500, MCLEAN, VA, 22102-3833, US

(21) Appl. No.: 10/970,725

(22) Filed: Oct. 22, 2004

Related U.S. Application Data
(60) Provisional application No. 60/512,853, filed on Oct. 22, 2003.

(51) Int. Cl. A61K 31/13; A61K 31/135

(52) U.S. Cl. 514/649; 514/660; 514/674

(57) ABSTRACT

A composition, method and article of manufacture for the amelioration of pain comprising: a polyamine having the formula:
R₁R₂N¹—A—[N²R₃—B]ₐ—[N³R₄—C]_{b}—N⁴R₅R₆ wherein:
R₁-R₆ may be the same or different and are alkyl, aryl, aryl alkyl, cycloalkyl, optionally having an alkyl chains interrupted by at least one etheric oxygen atom, or hydrogen;
N¹-N⁴ are nitrogen atoms capable of protonation at physiological pH's; a and b may be the same or different and are integers from 0 to 4; A, B and C may be the same or different and are bridging groups which effectively maintain the distance between the nitrogen atoms such that the polyamine: (i) is capable of uptake by a target cell upon administration of the polyamine to a human or non-human animal; and (ii) upon uptake by the target cell, competitively binds via an electrostatic interaction between the positively charged nitrogen atoms to substantially the same biological counter-anions as the intra-cellular natural polyamines in the target cell; the polyamine, upon binding to the biological counter-anion in the cell, functions to alleviate pain; a mixture of the amines; a derivative, salt or complex of the amine wherein the derivative, salt or complex former is physiologically acceptable and the formation of the salt, derivative or complex does not materially affect the pain amelioration properties of the amine; a mixture of the derivatives, salts and/or complexes, or a prodrug that provides the amine, mixture of the amines, the derivative, salt or complex or mixture of the derivatives, salts and/or complexes.

### FIELD OF THE INVENTION

The present invention relates to novel analgesic pharmaceutical compositions and methods for the amelioration of visceral pain.

### BACKGROUND OF THE INVENTION

The opioids are a group of drugs, both natural and synthetic, that are employed primarily as centrally-acting analgesics and are opium or morphine-like in their properties (Gilman et al., 1980, GOODMAN AND GILMAN'S. THE PHARMACOLOGICAL BASIS OF THERAPEUTICS, Chapter 24:494-534, Pub. Pergamon Press; hereby incorporated herein by reference). The opioids include morphine and morphine-like homologs, including, e.g., the semisynthetic derivatives codeine (methylmorphine) and hydrocodone (dihydrocodeinone) among many other such derivatives. Morphine and related opioids exhibit agonist activity at the central nervous system or CNS (referring to the brain and spinal cord) (mu) opioid receptors as well as showing affinity for the delta and kappa opioid receptors, to produce a range of effects including analgesia, drowsiness, changes in mood and mental clouding. In addition to potent analgesic effects, the morphine-related opioids may also cause a number of undesirable side effects, including, for example, respiratory depression, nausea, vomiting, dizziness, mental clouding, dysphoria, pruritus, constipation, increased biliary tract pressure, urinary retention and hypotension. The development of tolerance to the opioid drugs and the risk of chemical dependence and abuse for these drugs is another undesirable side effect.

Morphine, which has been considered the prototypic opioid analgesic, has been available in many dosage forms, including immediate release oral dosage forms, and more recently, formulated into 12 hour controlled release formulations (e.g., MS Contin.RTM. tablets, commercially available from Purdue Frederick Company). Other opioid analgesics have been available as immediate release oral dosage forms, such as hydromorphone (e.g., Dilaudid.RTM., commercially available from Knoll Pharmaceuticals). More recently, another controlled release opioid analgesic, oxycodone, has become available (OxyContin.RTM., commercially available from Purdue Pharma). There are, of course, many other oral formulations of immediate release and sustained release opioids which are commercially available throughout the world.

The use of various monoamines to alleviate pain is known in the prior art. For example, U.S. Pat. No. 6,710,087 discloses the treatment of neuropathic pain with sibutramine, [N-1-[1-(4-chlorophenyl)cyclobutyl]-3-methylbutyl]-N,N-dimethylamine]. It is disclosed in U.S. Pat. No. 6,376,553 to treat lower back pain with N,N-dimethyl-1-[1-(4-chlorophenyl)cyclobutyl]-3-methylbutyl amine hydrochloride. U.S. Pat. No. 5,688,830 indicates that 2-(2,6-dimethylphenoxy)-1-methylethyl-ethylamine is useful for the treatment of pain. Other U.S. patents disclosing the use of monoamine compounds to alleviate pain include U.S. Pat. Nos. 6,642,257; 5,843,942 and 5,063,231.

Polyamines, including spermine, spermidine, and putrescine, are known to stabilize polyanionic macromolecules of proteoglycans [Conroy et al., Biochem. J., 162:347-350 (1977)] and thus protect proteolytic and glycolytic enzymes. These polyamines also known to have an anti-inflammatory effect [Bird et al., Agents Actions, 13:342-347 (1983); Oyangui, Agents Actions, 14:228-237 (1984)], probably as a scavenger of oxygen-related products [Kafy et al., Agents Actions, 18:555-559 (1986)], and have an analgesic effect [Bird et al., supra; Oyangui, supra].

It is disclosed in U.S. Patent Publication No. 2004/0039057 that certain aliphatic polyamines are effective as topical analgesics. Among the polyamines disclosed as effective analgesics are putrescine, cadaverine, spermine and/or spermidine.

U.S. Pat. No. 6,121,326 discloses that various substituted alkyltetramine derivatives are effective tachykinin antagonists and are useful as anti-inflammatory or analgesic agents. The tetraamines are described as the reaction products of spermine and certain carboxylic acids.

It is an object of the invention to provide novel compositions and methods for the treatment of neuropathic pain involving the use of certain polyamines.

### SUMMARY OF THE INVENTION

It is an object of the invention to provide novel analgesic pharmaceutical compositions and methods for relieving pain that are not subject to the above-noted disadvantages associated with the opioid analgesics.

One embodiment of the invention relates to a method for ameliorating pain in a human or non-human mammal suffering therefrom comprising administering to the mammal an effective amount of an antinociceptive polyamine having the formula:
R₁R₂N¹—A—[N²R₃—B]ₐ—[N³R₄—C]_{b}—N⁴R₅R₆
wherein:
R₁-R₆ may be the same or different and are alkyl, aryl, aryl alkyl, cycloalkyl, optionally having a chain interrupted by at least one etheric oxygen atom, or hydrogen;
N¹- N⁴ are nitrogen atoms capable of protonation at physiological pH's;
a and b may be the same or different and are integers from 0 to 4;
A, B and C may be the same or different and are bridging groups which effectively maintain the distance between the nitrogen atoms such that the polyamine:
   (i) is capable of uptake by a target cell upon administration of the polyamine to a human or non-human animal; and
   (ii) upon uptake by the target cell, competitively binds via an electrostatic interaction between the positively charged nitrogen atoms to substantially the same biological counter-anions as the intra-cellular natural polyamines in the target cell;
the polyamine, upon binding to the biological counter-anion in the cell, functions to alleviate pain;

a mixture of the amines; a derivative, salt or complex of the amine wherein the derivative, salt or complex former is physiologically acceptable and the formation of the salt, derivative or complex does not materially affect the pain amelioration properties of the amine; a mixture of the derivatives, salts and/or complexes, or a prodrug that provides the amine, mixture of the amines, the derivative, salt or complex or mixture of the derivatives, salts and/or complexes.

Another embodiment of the invention concerns a pharmaceutical composition adapted for ameliorating pain in a human or non-human mammal suffering therefrom comprising an effective amount of an antinociceptive polyamine having the formula:
R₁R₂N¹—A—[N²R₃—B]ₐ—[N³R₄—C]_{b}—N⁴R₅R₆
wherein:
R₁-R6 may be the same or different and are alkyl, aryl, aryl alkyl, cycloalkyl, optionally having an alkyl chains interrupted by at least one etheric oxygen atom, or hydrogen;
N¹- N⁴ are nitrogen atoms capable of protonation at physiological pH's;
a and b may be the same or different and are integers from 0 to 4;
A, B and C may be the same or different and are bridging groups which effectively maintain the distance between the nitrogen atoms such that the polyamine:
   (i) is capable of uptake by a target cell upon administration of the polyamine to a human or non-human animal; and
   (ii) upon uptake by the target cell, competitively binds via an electrostatic interaction between the positively charged nitrogen atoms to substantially the same biological counter-anions as the intra-cellular natural polyamines in the target cell;
the polyamine, upon binding to the biological counter-anion in the cell, functions to alleviate pain;

a mixture of the amines; a derivative, salt or complex of the amine wherein the derivative, salt or complex former is physiologically acceptable and the formation of the salt, derivative or complex does not materially affect the pain amelioration properties of the amine; a mixture of the derivatives, salts and/or complexes, or a prodrug that provides the amine, mixture of the amines, the derivative, salt or complex or mixture of the derivatives, salts and/or complexes.

Another embodiment of the invention relates to an article of manufacture comprising packaging material and a pharmaceutical agent contained within the packaging material, wherein the pharmaceutical agent is effective for the treatment of a subject suffering from pain, and wherein the packaging material comprises a label which indicates that the pharmaceutical agent can be used for ameliorating pain, and wherein the pharmaceutical agent is an antinociceptive polyamine having the formula:
R₁R₂N¹—A—[N²R₃—B]ₐ—[N³R₄—C]_{b}—N⁴R₅R₆
wherein:
R₁-R₆ may be the same or different and are alkyl, aryl, aryl alkyl, cycloalkyl, optionally having an alkyl chains interrupted by at least one etheric oxygen atom, or hydrogen;
N¹-N⁴ are nitrogen atoms capable of protonation at physiological pH's;
a and b may be the same or different and are integers from 0 to 4;
A, B and C may be the same or different and are bridging groups which effectively maintain the distance between the nitrogen atoms such that the polyamine:
   (i) is capable of uptake by a target cell upon administration of the polyamine to a human or non-human animal; and
(ii) upon uptake by the target cell, competitively binds via an electrostatic interaction between the positively charged nitrogen atoms to substantially the same biological counter-anions as the intra-cellular natural polyamines in the target cell;
the polyamine, upon binding to the biological counter-anion in the cell, functions to alleviate pain;

a mixture of the amines; a derivative, salt or complex of the amine wherein the derivative, salt or complex former is physiologically acceptable and the formation of the salt, derivative or complex does not materially affect the pain amelioration properties of the amine; a mixture of the derivatives, salts and/or complexes, or a prodrug that provides the amine, mixture of the amines, the derivative, salt or complex or mixture of the derivatives, salts and/or complexes.

### BRIEF DESCRIPTION OF THE DRAWINGS

FIGS. 1a, 1b, 2a and 2b graphically depict test results of polyamines of the invention.

### DETAILED DESCRIPTION OF THE INVENTION

In the polyamines of the invention, as described in the above structural formula, R₁-R₆ may be alkyl, e.g., methyl, ethyl, n-propyl, isopropyl, n-butyl, isobutyl, sec-butyl, tert-butyl; aryl, e.g., phenyl, p-tolyl, 2,4,6-trimethylphenyl; aryl alkyl, e.g., benzyl, α-phenethyl, β-phenethyl; cycloalkyl, e.g., cyclohexyl, cyclobutyl, cyclopentyl, cycloheptyl; any of the foregoing wherein the alkyl chain is interrupted by etheric oxygen, e.g., CH₃O(CH₂)₂—, CH₃O(CH₂)₂O(CH₂)₂—, CH₃O(CH₂)₂O(CH₂)₂O(CH₂)₂—; or hydrogen.

Except where R₁-R₆ are hydrogen or etheric substituents, each are hydrocarbyl and may have from about 1 to about 12 carbon atoms, it being understood that the size of the substituents will be tailored in each case to ensure that the polyamine is capable of uptake by the target cell and, upon uptake, will competitively bind with the intracellular counter-anions as described above.

The bridging groups A, B and C may be the same or different and may be alkylene having 1-8 carbon atoms, e.g., methylene, trimethylene, tetramethylene, pentamethylene; branched alkylene, e.g., —CH(CH₃)CH₂CH₂—, —CH₂CH(CH₃)CH₂—, —CH(CH₃)CH₂CH₂—, —CH₂CH(CH₃)CH₂CH₂—; arylalkylene, e.g., —CH(Ph)CH₂CH₂—, —CH₂CH(Ph)CH₂—, CH(Ph)CH₂CH₂CH₂—, —CH₂CH(Ph)CH₂CH₂—, optionally substituted by OH at a carbon atom not alpha to a N group, e.g., —CH₂—CHOH—CH₂—; cycloalkylene, e.g., cyclohexylene, cis- and trans-1,3-cyclohexylene, 1,4-cyclohexylene, 1,3-cyclopentylene; heterocyclic groups which incorporate within the ring one of the nitrogen atoms of the polyamine [e.g., 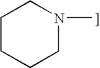
it being understood that the heterocyclic nitrogen group may be located at the terminal end(s) or within the interior of the polyamine. Those skilled in the art will appreciate that it is only necessary that the bridging groups be selected so as to ensure uptake by the cell and competitive binding to the intracellular counter-anion as described above.

The preferred polyamines for use in the practice of the present invention include dihydroxydiethylnorspermine [(OH)₂DENSPM], having the formula:
CH₃CH₂—NH—CH₂—CHOH—CH₂—NH—(CH₂)₃—NH—CH₂—CHOH—CH₂—NH—CH₂CH₃;
N¹N¹¹-dibutylspermine [DBSPM]; CH₃CH₂—NH—(CH₂)₃—NH—cyclohexyl—NH₂ [cyclohexyl fragment], having the formula: 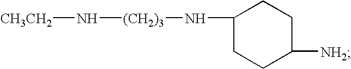
CH₃CH₂—NH—(CH₂)₃—NH-cyclohexyl, having the formula: 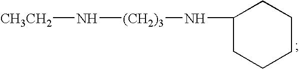
CH₃CH₂—NH—(CH₂)₄—NH-cyclohexyl, having the formula: 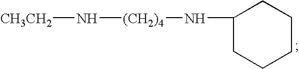
N,N′-bis[3-(ethylamino)propyl]-trans-1,4-cyclohexanediamine [CHX(3,4,3)-trans], having the formula: 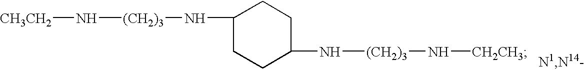
diethylhomospermine [DEHSPM]; N¹,N¹¹-diethylnorspermine [DENSPM]; N,N′-bis(4-piperidinyl)-1,4-butanediamine [PIP(3,4,3)], having the formula: 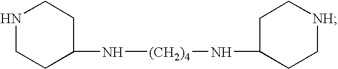
N¹,N¹²-diethylspermine [DESPM] and N¹N¹²-diisopropylspermime [DIPSPM]. All of the polyamines and chemical compounds described herein are known in the prior art. The polyamines are described as well as methods for their preparation in U.S. Pat. Nos. 6,342,534; 6,297,287; 6,274,630; 6,262,125; 6,235,794; 6,184,232; 6,147,262; 6,034,139; 5,962,533; 5,866,613; 5,827,894; 5,677,352; 5,510,390; 5,462,970; 5,455,277; 5,393,757; 5,342,945; and Bergeron R J, McManis J S, Liu C Z, Feng Y, Weimar W R, Luchetta G R, Wu Q, Ortiz-Ocasio J, Vinson J R T, Kramer D, Porter C: Antiproliferative properties of polyamine analogues: A structure-activity study, J Med Chem 37:3464-3476, 1994; Bergeron R J, Feng Y, Weimar W R, McManis J S, Dimova H, Porter C, Raisler B, Phanstiel 0: A comparison of structure-activity relationships between spermidine and spermine analogue antineoplastics. J Med Chem 40:14751494, 1997; Bergeron R J, McManis J S, Weimar W R, Schreier K M, Gao F, Wu Q, Ortiz-Ocasio J, Luchetta G R, Porter C, Vinson J R T: The role of charge in polyamine analogue recognition. J Med Chem 38:2278-2285, 1995; and Bergeron et al, J. Med Chem. 44:232-244 (2001), the entire contents and disclosures of all and each of which are incorporated herein by reference.

The results set forth in Table 1 demonstrate that N,N′-bis[3-(ethylamino)propyl]-trans-1,4-cyclohexanediamine [CHX(3,4,3)-trans] and [(OH)₂DENSPM] are most effective in the murine visceral pain model after s.c. administration of doses less than 0.005 mg/kg. DBSPM and DENSPM are also effective at subcutaneous doses 0.1-0.4 mg/kg. The results also indicate that N¹,N¹⁴-diethylhomospermine [DEHSPM] and N¹,N¹¹-diethylnorspermine [DENSPM] are also highly effective and N,N′-bis(4-piperidinyl)-1,4-butanediamine [PIP(3,4,3)]; N¹,N¹²-diethylspermine [DESPM] and N¹N¹²-diisopropylspermime [DIPSPM], less so.

Visceral Pain: Experiments have demonstrated that some polyamine analogues suppress visceral nociception in a rodent model. Dose-response relationships for the polyamines listed in Table 1 were performed in the mouse i.p. acetic acid test. These data demonstrated that CHX(3,4,3)-trans was more than three orders of magnitude more potent than morphine at inhibiting visceral nociception. DEHSPM, on the other hand, was equipotent to morphine. Most importantly, the polyamine analogues suppressed visceral nociception in the mice without overt cognitive effects, whereas morphine produced stereotypical opiate induced hyperactivity and Straub tail (Aceto M D, McKean D B, Pearl J. Effects of opiates and opiate antagonists on the Straub tail reaction in mice. Br J Pharmacol. 1969;36:225-239; Gupta M L, Nath R, Gupta T K, Gupta G P. A study of central neurotransmitter mechanisms in morphine-induced ‘Straub reaction’ in mice: Role of central dopamine receptors. Clin Exp Pharmacol Physiol. 1988;15:727-732.)

This finding is consistent with the lack of cognitive effects of DEHSPM in human clinical trials and indicates that polyamine analogs will be effective for ameliorating visceral pain without the side effect profile of opiate narcotics.

Assessment of Candidate Agents in a Murine Visceral Pain Model: There is an extensive literature on visceral pain in the acetic acid mouse model (Bidlack J M, Cohen D J, McLaughlin J P, Lou R, Ye Y, Wentland M P. 8-carboxamidocyclazocine: A long-acting, novel benzomorphan. J Pharmacol Exp Ther. 2002;302:374-380; Reichert J A, Daughters R S, Rivard R, Simone D A. Peripheral and preemptive opioid antinociception in a mouse visceral pain model. Pain. 2001;89:221-227; Farzin D, Asghari L, Nowrouzi M. Rodent antinociception following acute treatment with different histamine receptor agonists and antagonists. Pharmacol Biochem Behav. 2002;72:751-760; Hinschberger A, Butt 5, Lelong V, Boulouard M, Dumuis A, Dauphin F, Bureau R, Pfeiffer B, Renard P, Rault S. New benzo[h][1,6]naphthyridine and azepino[3,2-c]quinoline derivatives as selective antagonists of 5-ht4 receptors: Binding profile and pharmacological characterization. J Med Chem. 2003;46:138-147).

The mice receive either an s.c. or i.p. injection (0.3 mL) of either saline or a test drug at varying doses. Fifteen to thirty minutes following the treatment, the animals receive an i.p. injection of 0.6% acetic acid in saline (sterile, 0.3 mL). The mice are then placed into holding cages and observed for 30 mm. The number of stretches the animals make following the acetic acid injections are counted. A stretch is identified by the animal extending its hind legs well behind its body. This posture is rarely seen in a non-injected animal. After the experimental period, the number of stretches are graphed vs. the dose to determine both the dose-response relationship and the dose at which the number of stretches during the observation period is significantly different from saline controls.

TABLE 1
Activity of polyamines on
Stretching in Murine i.p. Acetic Acid Test

| Agent | MED^{a} (mg/kg s.c.) |
|---|---|
| CHX(3,4,3)-trans | 0.0037 |
| (HO)₂DENSPM | 0.0037 |
| DBSPM | 0.12 |
| DENSPM | 0.37 |

^{a}Minimum dose at which the number of stretches during the observation period is significantly different from saline controls.

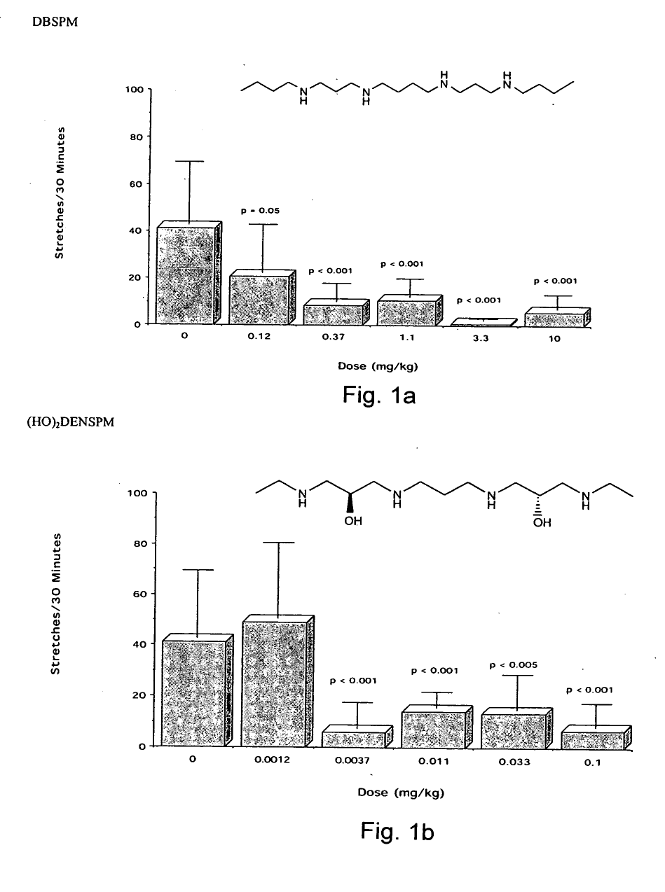

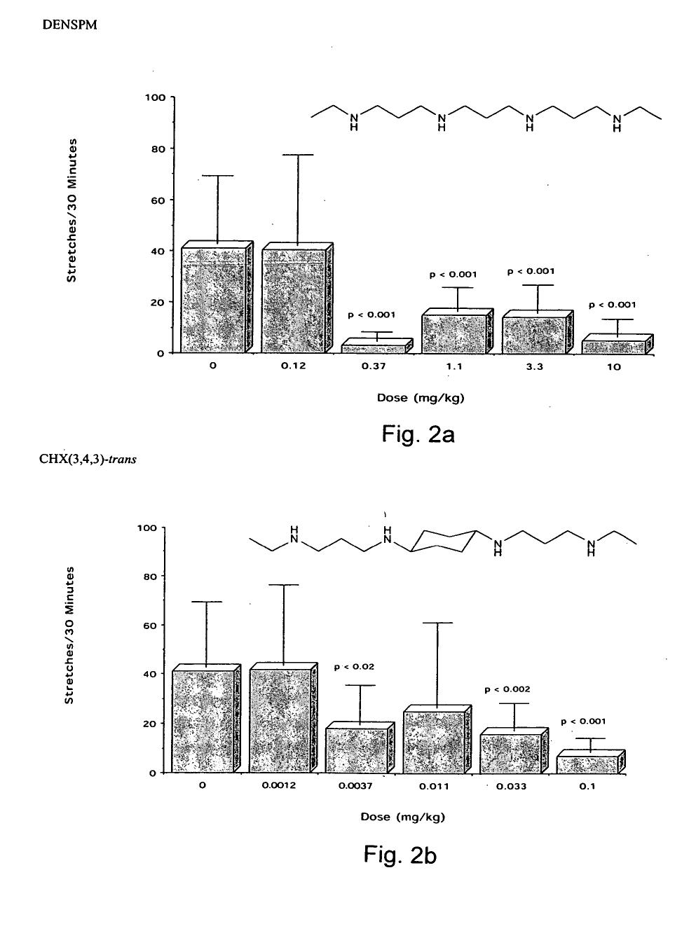

Results of stretch tests employing polyamines of the invention are also set forth in FIGS. 1a, 1b, 2a and 2b.

The skilled artisan will appreciate that pain is a heterogeneous disorder. In the methods and compositions according to the invention, the term “pain” shall refer to all types of pain, including acute and persistent pain. Preferably, the term shall refer to persistent pains, such as, but not limited to, neuropathic pain, diabetic neuropathy, fibromyalgia, pain associated with somatoform disorders, arthritic pain, cancer pain, neck pain, shoulder pain, back pain, cluster headaches, tension-type headache, migraine, herpes neuralgia, phantom limb pain, central pain, dental pain, NSAID-resistant pain, visceral pain, surgical pain, post-operative pain, bone injury pain, pain during labor and delivery, pain resulting from bums, including sunburn, post-partum pain, angina pain, and genitourinary tract-related pain including cystitis. The term persistent pain shall also preferably refer to nociceptive pain or nociception.

The articles of manufacture, method and composition of the present invention are predicated on administering to a subject (human or animal) suffering from pain an effective amount of one or more of the compounds described herein as being effective therefore. Administration may be accomplished either therapeutically or prophylactically by means of pharmaceutical compositions which are prepared by techniques well known in the pharmaceutical sciences. The polyamines of the present invention may be used either solely or jointly in pharmaceutically effective amounts for treating animals or humans. The polyamines of the invention can be used either solely or jointly together in pharmaceutically acceptable amounts with pharmaceutically effective amounts of other pharmaceutically active components in pharmaceutical compositions or preparations.

While the compounds of the invention are preferably administered orally, they may also be administered by a variety of other routes such as transdermally, subcutaneously, intranasally, intrarectally, intramuscularly and intravenously. The present invention is also directed to pharmaceutical compositions which include at least one compound as described above in association with one or more pharmaceutically acceptable diluents, excipients or carriers therefor. In making the pharmaceutical compositions of the present invention, one or more compounds will usually be mixed with, diluted by or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 60% by weight of active compound, soft and hard gelatin capsules, suppositories, sterile injectable solutions and sterile packaged powders. In the case of injections, it is possible to prepare solutions or suspensions of one or more polyamines of the present invention in pharmaceutically acceptable carriers such as an aqueous or nonaqueous solvent. Examples of solvents which may be used are distilled water for injection, physiological saline solution, Ringer's solution, plant oil, synthetic fatty acid glycerides, higher fatty acid esters, propylene glycol, etc.

Some examples of suitable carriers, excipients and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propyl-hydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide rapid, sustained or delayed release of the active ingredient after administration to the patient by employing procedures well known in the art.

The dose of the compound is that amount effective to prevent occurrence of pain or to treat existing pain from which the patient suffers. By “effective amount,” “therapeutic amount” or “effective dose” is meant that amount sufficient to elicit the desired pharmacological or therapeutic effects, thus resulting in effective prevention or treatment of pain. Prevention of pain is manifested by a prolonging or delaying of the onset of the symptoms of pain. Treatment of pain is manifested by a decrease in the symptoms associated therewith or an amelioration of the recurrence of the pain.

The effective dose may vary, depending upon factors such as the condition of the patient, the severity of the symptoms of pain and the manner in which the pharmaceutical composition is administered. The compositions are formulated, preferably in a unit dosage form, such that each dosage contains from about 0.006 to about 12,000 mg., more usually about 0.06 to about 6,000 mg., of the active ingredient. The term “unit dosage form” refers to physically discrete units suitable as unitary dosages for human subjects and other mammals, each unit containing a predetermined quantity of active material calculated to produce the desired therapeutic effect, in association with one or more of the above-described suitable pharmaceutical diluents, excipients or carriers.

The compounds are effective over a wide dosage range in treating pain. Thus, as used herein, the term “effective amount” refers to a dosage range of from about 0.006 to about 500 mg/kg of body weight per day. In the treatment of adult humans, the range of about 0.06 to about 250 mg/kg, in single or divided doses, is preferred. However, it will be understood that the amount of compound actually administered will be determined by a physician in light of the relevant circumstances, including (1) the condition to be treated, (2) the choice of compound to be administered, (3) the chosen route of administration, (4) the age, weight and response of the individual patient, and (5) the severity of the patient's symptoms. Therefore, the above dosage ranges are not intended to limit the scope of the invention in any way.

By “active ingredient” is meant a polyamine as described herein or a salt thereof with a pharmaceutically acceptable acid. By “salt” is meant an addition salt between the polyamine of the invention and a sufficient amount of pharmacologically appropriate acid, such as hydrochloric, sulfuric, phosphoric, acetic, butyric, citric, maleic, lactic, valeric, tartaric, gluconic, succinic and the like, made by conventional chemical means.

1. A method for ameliorating pain in a human or non-human mammal suffering therefrom comprising administering to the mammal an effective amount of an antinociceptive polyamine having the formula:
R₁R₂N¹—A—[N²R₃—B]ₐ—[N³R₄—C]_{b}—N⁴R₅R₆
wherein:
R₁-R6 may be the same or different and are alkyl, aryl, aryl alkyl, cycloalkyl, optionally having an alkyl chains interrupted by at least one etheric oxygen atom, or hydrogen;
N¹-N⁴ are nitrogen atoms capable of protonation at physiological pH's;
a and b may be the same or different and are integers from 0 to 4;
A, B and C may be the same or different and are bridging groups which effectively maintain the distance between said nitrogen atoms such that said polyamine:
(i) is capable of uptake by a target cell upon administration of said polyamine to a human or non-human animal; and
(ii) upon uptake by said target cell, competitively binds via an electrostatic interaction between the positively charged nitrogen atoms to substantially the same biological counter-anions as the intra-cellular natural polyamines in the target cell; said polyamine, upon binding to said biological counter-anion in the cell, functions to alleviate pain;
a mixture of said amines; a derivative, salt or complex of said amine wherein the derivative, salt or complex former is physiologically acceptable and the formation of said salt, derivative or complex does not materially affect the pain amelioration properties of said amine; a mixture of said derivatives, salts and/or complexes, or a prodrug that provides said amine, mixture of said amines, said derivative, salt or complex or mixture of said derivatives, salts and/or complexes.

2. The method of claim 1 wherein said polyamine is dihydroxydiethylnorspermine, having the formula:
CH₃CH₂—NH—CH₂—CHOH—CH₂—NH—(CH₂)₃—NH—CH₂—CHOH—CH₂—NH—CH₂CH₃

3. The method of claim 1 wherein said polyamine is N¹N¹¹-dibutylspermine.

4. The method of claim 1 wherein said polyamine is CH₃CH₂—NH—(CH₂)₃—NH—cyclohexyl—NH₂ having the formula:

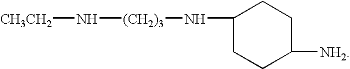

5. The method of claim 1 wherein said polyamine is CH₃CH₂—NH—(CH₂)₃—NH—cyclohexyl.

6. The method of claim 1 wherein said polyamine is CH₃CH₂—NH—(CH₂)₄—NH—cyclohexyl.

7. The method of claim 1 wherein said polyamine is N,N′-bis[3-(ethylamino)propyl]-trans-1,4-cyclohexanediamine having the formula

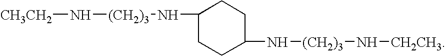

8. The method of claim 1 wherein said polyamine is N¹,N¹⁴-diethylhomospermine.

9. The method of claim 1 wherein said polyamine is N¹,N¹¹-diethylnorspermine

10. The method of claim 1 wherein said polyamine N,N′-bis(b 4-piperidinyl)-1,4-butanediamine having the formula

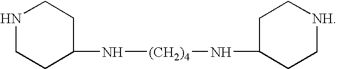

11. The method of claim 1 wherein said polyamine is N¹,N¹²-diethylspermine.

12. The method of claim 1 wherein said polyamine is N¹N¹²-diisopropylspermime.

13. A composition for the for ameliorating pain in a human or non-human mammal suffering therefrom comprising (1) an effective amount of an antinociceptive amine having the formula: R₁R₂N¹—A—[N²R₃—B]ₐ—[N³R₄—C]_{b}—N⁴R₅R₆
wherein:
R₁-R₆ may be the same or different and are alkyl, aryl, aryl alkyl, cycloalkyl, optionally having an alkyl chains interrupted by at least one etheric oxygen atom, or hydrogen;
N¹-N⁴ are nitrogen atoms capable of protonation at physiological pH's;
a and b may be the same or different and are integers from 0 to 4;
A, B and C may be the same or different and are bridging groups which effectively maintain the distance between said nitrogen atoms such that said polyamine:
(i) is capable of uptake by a target cell upon administration of said polyamine to a human or non-human animal; and
(ii) upon uptake by said target cell, competitively binds via an electrostatic interaction between the positively charged nitrogen atoms to substantially the same biological counter-anions as the intra-cellular natural polyamines in the target cell, said polyamine, upon binding to said biological counter-anion in the cell, functions to alleviate pain;
a mixture of said amines; a derivative, salt or complex of said amine wherein the derivative, salt or complex former is physiologically acceptable and the formation of said salt, derivative or complex does not materially affect the pain amelioration properties of said amine; a mixture of said derivatives, salts and/or complexes, or a prodrug that provides said amine, mixture of said amines, said derivative, salt or complex or mixture of said derivatives, salts and/or complexes, and (2) a pharmacologically acceptable carrier therefore.

14. The composition of claim 13 wherein said polyamine is dihydroxydiethylnorspermine, having the formula:
CH₃CH₂—NH—CH₂—CHOH—CH₂—NH—(CH₂)₃—NH—CH₂—CHOH—CH₂—NH—CH₂CH₃

15. The composition of claim 13 wherein said polyamine is N¹N¹¹-dibutylspermine.

16. The composition of claim 13 wherein said polyamine is CH₃CH₂—NH—(CH₂)₃—NH—cyclohexyl—NH₂ having the formula

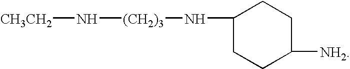

17. The composition of claim 13 wherein said polyamine is CH₃CH₂—NH—(CH₂)₃—NH—cyclohexyl.

18. The composition of claim 13 wherein said polyamine is CH₃CH₂—NH—(CH₂)₄—NH—cyclohexyl.

19. The composition of claim 13 wherein said polyamine is N,N′-bis[3-(ethylamino)propyl]-trans-1,4-cyclohexanediamine having the formula

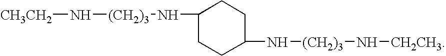

20. The composition of claim 13 wherein said polyamine is N¹,N¹⁴-diethylhomospermine.

21. The composition of claim 13 wherein said polyamine is N¹,N¹¹-diethylnorspermine

22. The composition of claim 13 wherein said polyamine N,N′-bis(4-piperidinyl)-1,4-butanediamine having the formula:

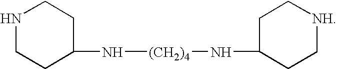

23. The composition of claim 13 wherein said polyamine is N¹,N¹²-diethylspermine.

24. The composition of claim 13 wherein said polyamine is N¹N¹²-diisopropylspermime.

25. An article of manufacture comprising packaging material and a pharmaceutical agent contained within said packaging material, wherein said pharmaceutical agent is effective for the treatment of a subject suffering from pain, and wherein said packaging material comprises a label which indicates that said pharmaceutical agent can be used for ameliorating pain, and wherein said pharmaceutical agent is a polyamine having the formula:
R₁R₂N¹—A—[N²R₃—B]ₐ—[N³R₄—C]_{b}—N⁴R₅R₆
wherein:
R₁-R₆ may be the same or different and are alkyl, aryl, aryl alkyl, cycloalkyl, optionally having an alkyl chains interrupted by at least one etheric oxygen atom, or hydrogen;
N¹-N⁴ are nitrogen atoms capable of protonation at physiological pH's;
a and b may be the same or different and are integers from 0 to 4;
A, B and C may be the same or different and are bridging groups which effectively maintain the distance between said nitrogen atoms such that said polyamine:
(i) is capable of uptake by a target cell upon administration of said polyamine to a human or non-human animal; and
(ii) upon uptake by said target cell, competitively binds via an electrostatic interaction between the positively charged nitrogen atoms to substantially the same biological counter-anions as the intra-cellular natural polyamines in the target cell;
said polyamine, upon binding to said biological counter-anion in the cell, functions to alleviate pain;
a mixture of said amines; a derivative, salt or complex of said amine wherein the derivative, salt or complex former is physiologically acceptable and the formation of said salt, derivative or complex does not materially affect the pain amelioration properties of said amine; a mixture of said derivatives, salts and/or complexes, or a prodrug that provides said amine, mixture of said amines, said derivative, salt or complex or mixture of said derivatives, salts and/or complexes.

26. The article of claim 24 wherein said polyamine is dihydroxydiethylnorspermine, having the formula:
CH₃CH₂—NH—CH₂—CHOH—CH₂—NH—(CH₂)₃—NH—CH₂—CHOH—CH₂—NH—CH₂CH₃

27. The article of claim 24 wherein said polyamine is N¹N¹¹-dibutylspermine.

28. The article of claim 24 wherein said polyamine is CH₃CH₂—NH—(CH₂)₃—NH—cyclohexyl—NH₂ having the formula:

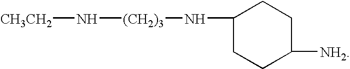

29. The article of claim 24 wherein said polyamine is CH₃CH₂—NH—(CH₂)₃—NH—cyclohexyl.

30. The article of claim 24 wherein said polyamine is CH₃CH₂—NH—(CH₂)₄—NH—cyclohexyl.

31. The article of claim 24 wherein said polyamine is N,N′-bis[3-(ethylamino) propyl]-trans-1,4-cyclohexanediamine having the formula:

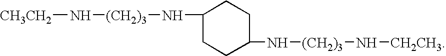

32. The article of claim 24 wherein said polyamine is N¹,N¹⁴-diethylhomospermine.

33. The article of claim 24 wherein said polyamine is N¹,N¹¹-diethylnorspermine.

34. The article of claim 24 wherein said polyamine N,N′-bis(4-piperidinyl)-1,4-butanediamine having the formula:

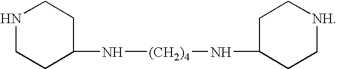

35. The article of claim 24 wherein said polyamine is N¹,N¹²-diethylspermine.

36. The article of claim 24 wherein said polyamine is N¹N¹²-diisopropylspermime.
